(19) United States
(12) Patent Application Publication
(10) Pub. No.: US 2013/0345661 A1
(43) Pub. Date: Dec. 26, 2013

(54) Skin Treatment Device

(71) Applicant: Franklin J. Chang, Pasadena, CA (US)

(72) Inventor: Franklin J. Chang, Pasadena, CA (US)

(21) Appl. No.: 13/533,719

(22) Filed: Jun. 26, 2012

(51) Int. Cl. A61M 37/00; A61N 1/30

(52) U.S. Cl. 604/501; 604/20

(57) ABSTRACT

A device and methods for increasing the permeability of the skin's surface to fluid and/or drug delivery is described. The device comprises an abrasive media to remove the outer layer of the stratum corneum, while at the same time applying an electric current that stimulates the skin under the stratum corneum, and delivers fluids from a supply reservoir. The device may also have a vacuum function which evacuates fluid and skin debris from the surface of the skin and delivers the evacuated fluid and skin debris to a waste (or collection) container.

### BACKGROUND

Current techniques for superficial skin resurfacing, known as microdermabrasion, treat the outer epidermal layer of the skin by removing the superficial layer to induce the body's own natural wound healing response. It is known in the art to couple microdermabrasion with fluid delivery to enhance therapeutic effects. However, combined microdermabrasion/fluid delivery treatments are hindered by the protective barrier function of the stratum corneum which limits the depth of penetration and absorption to the surface of the skin when drugs and/or fluids are applied to the skin.

Other techniques for skin enhancing include transdermal drug delivery employing an electrical current (e.g., skin electroporation) are known. However, these techniques have limited results based on: 1) the lack of an efficient fluid supply/return system using a vacuum; 2) the impedance of the stratum corneum which limits the efficacy of the current technologies of electrical penetration of drugs and/or fluids; and 3) the optimal permeation structure of the skin occurs during application of an electrical current and only lasts a few seconds after application of the electrical pulse.

Known technologies for delivery of an electro-current to the skin suffer from one or more of the following deficiencies which lead to the limited results, including, lack an efficient fluid supply/return using a vacuum; an inability to simultaneously apply fluid and electro-current to the skin; and means to lower the impedance of the stratum corneum.

Accordingly, there is a need for a skin resurfacing and enhancement system with enhanced fluid delivery/fluid return capacity which also improves the permeation structure of the skin.

### SUMMARY

According to the present invention, devices and methods for a combination treatment of the top and bottom layer of a skin surface are described. The device comprises transdermal drug and/or fluid delivery with electrodes providing electric current to stimulate the skin, and an abrasive tip to peel the top layer of skin simultaneously applied to the skins surface. According to one embodiment of the present invention, a skin treatment device that combines a fluid delivery system, an abrasive tip, and an electric current delivery probe in one handle is described. Preferably the device further comprises a vacuum source for removal of fluid and skin debris from the surface of the skin.

The device and methods described herein allow for the simultaneous deep penetration of fluid through the skin by applying an electric current and an abrasive media in the working end of the device to increase skin's permeability. According to alternate embodiments, techniques known as electroporation, ultrasound, and other electrical induced therapies, etc., which use electric currents to go deeper past the stratum corneum to stimulate cells underneath the skin may be employed in the device. The combination of the electrical induced therapies and microdermabrasion create aqueous pathways to increase the permeability of the drugs and/or fluids which are delivered from a supply and return reservoir by a vacuum system within the device. A pressure mechanism may also be employed as part of the device.

According to one embodiment, a device for treating a skin surface of a patient comprising a handle having a tip at the proximal end of the handle is provided. The tip has one or more electrodes and an abrading end portion which has an abrasive media and one or more apertures for fluid delivery. The device may also have a vacuum and a vacuum entry port located on the tip at the proximal end of the handle, where the vacuum entry port has one or more apertures for evacuating fluid and debris from the surface of the skin. According to another embodiment, the electrodes, abrading end portion and fluid delivery apertures are positioned on the tip of the handle, where each one individually may be on a removable tip or end structure. When the device has a plurality of removable structures, the end structures may also be separately removable and interchangeable.

In a preferred embodiment, the tip of the device has an outer structure having one or more electrodes and an intermediate structure having an abrading end portion, where the abrading end portion has an abrasive media. The tip of the device also has an inner structure which has one or more apertures for fluid delivery. Preferably, at least one of the structures is removable, and more preferably, each of the outer structure, intermediate structure, and inner structure are removable, and most preferably, at least one of the structures is disposable.

According to another embodiment, a method for treating a skin surface of a patient is provided. According to the method, first an abrasion device for treating a skin surface of a patient is selected, wherein the abrasion device comprises one or more electrodes, an abrading end portion having an abrasive media, and one or more apertures for fluid delivery. Next, the abrading end portion of the device is placed on the skin surface of the patient. The patient's skin is then treated by applying the abrasive media to the skin surface of the patient, delivering fluid to the skin surface of the patient, and applying an electrical current to the skin surface of the patient. The patient's skin is treated with abrasive media, fluid delivery, and current delivery in the order stated above, simultaneously, or another order. Vacuum may then be applied to the skin surface of the patient.

According to another embodiment, a kit for treating a skin surface of a patient is provided. The kit comprises a skin abrading device comprising a tip, wherein the tip has at least one current delivery tip having one or more electrodes, a plurality of abrading tips, wherein each abrading tip has an end portion with an abrasive media, and wherein the plurality of abrading tips are removable from the device and interchangeable, and a fluid delivery tip having one or more apertures for fluid delivery. Preferably, the tip further comprises a vacuum entry port and also preferably, each of the plurality of abrading tips has a grit size, and the grit size varies for each abrading tip.

### FIGURES

These and other features, aspects and advantages of the present invention will become better understood from the following description, appended claims, and accompanying figures where:

FIG. 1A shows a skin abrading device 100 according to one embodiment of the present invention;

FIG. 1B is partial side cut-away view of the device 100, shown in FIG. 1A, according to the present invention;

FIG. 2A is a top perspective view of the device 100, shown in FIG. 1A and FIG. 1B, showing the tip 104 of the device 100, according to the present invention;

FIG. 2B and FIG. 2C are alternate embodiments for the tip 104 of the device 100, according to another embodiment of the present invention;

FIG. 3A is a side view of one embodiment of the device 100, having a plurality of removable, exchangeable, and attachable tips according to another embodiment of the present invention;

FIG. 3B is a side view of another embodiment of one of the tips shown in FIG. 3A;

FIG. 4 is a partial side cut-away view of the device 100 having a wide-angle tip 104 according to another embodiment of the present invention;

FIG. 5A is a side view of another embodiment of the device 100, having a plurality of removable, exchangeable, and attachable tips, where the electrodes 108a, 108b, are concentric circles, according to another embodiment of the present invention;

FIG. 5B is a partial side cut-away view of the device 100 shown in FIG. 5A, having electrodes 108a, 108b, which are concentric circles, according to another embodiment of the present invention; and

FIG. 6A shows an alternate embodiment of the skin abrading device 100 according to another embodiment of the present invention, having a divided handle 102a and 102b; and

FIG. 6B is a cut-away view showing the divided handle 102a and 102b of FIG. 6A.

### DESCRIPTION

According to the present invention, a device for increasing the permeability of the skins surface to fluid and/or drug delivery is described. In general, permeation of drugs and/or fluids through the skin occurs at a slow rate, if at all. The stratum corneum acts as a barrier that limits the penetration of substances through the skin. Application of high-voltage pulses to the skin increases its permeability (electroporation) and enables the delivery of various substances into and through the skin. The application of electroporation to the skin has been shown to increase transdermal drug delivery, Moreover, electroporation, used alone or in combination with other enhancement methods, expands the range of drugs (small to macromolecules, lipophilic or hydrophilic, charged or neutral molecules) that can be delivered transdermally. The efficacy of transport depends on the electrical parameters and the physicochemical properties of drugs. The in vivo application of high-voltage pulses is well tolerated.

According to one embodiment of the invention, a device comprising an abrading surface, fluid delivery, current delivery; and fluid vaccuation is described. The device enhances fluid delivery through the stratum corneum by first delivering an abrasive media to the surface of the skin to prepare the skin for fluid delivery. Next, the device delivers fluid to the surface of the skin, with simultaneous current delivery (electroporation). The combination of skin abrasion, followed by simultaneous fluid delivery with electroporation allows for deep penetration of fluid through the skin by increasing the skin's permeability. In addition to enhancing fluid delivery through the stratum corneum, the device resurfaces the outer surface of the skin, removing dead skin cells and the outer layer of dermis, along with other superficial imperfections. Unlike known microdermabrasion devices, the results achieved with the device of the present invention will have enhanced and longer lasting results, namely, because skin enhancing fluids and drugs are delivered more deeply into the skin with the simultaneous electrooporation, and the electrical induced therapy itself has skin enhancing properties, such as increased collagen production, muscle tone, and overall skin elasticity and firmness.

The device and methods described herein have an efficient fluid supply/return for transdermal/topical delivery of skin enhancing drugs and medicaments. This feature of the invention has been found to be particularly important since presently known technologies use a gel which is applied to the skin which limits the penetration of effective ingredients because of the greater molecular weight of the gel. Macromolecule delivery through a liquid, which can be accomplished with the present invention, is accordingly more effective than prior art technologies which use a gel. The application of an abrasive as described in this invention solves this issue of lowering the impedance of the stratum corneum thus further improving drug delivery to the skin. Accordingly, the device and methods of the present invention, which include fluid delivery with electro-current and a vacuum source, enable simultaneous application of fluids containing skin enhancing drugs, with increased topical delivery through an abrading surface, to achieve the maximum effect. The abrading surface, which is applied to the skin preferably prior to fluid/drug delivery, increases topical drug delivery and penetration of the drug to the lower layers of the skin. These features of the invention are an improvement over prior art technologies which lack a fluid delivery and a vacuum source and more particularly in combination with an abrading surface and electro-current application to accomplish skin resurfacing and enhancement.

As used in this disclosure, the term “comprise” and variations of the term, such as “comprising” and “comprises,” are not intended to exclude other additives, components, integers or steps.

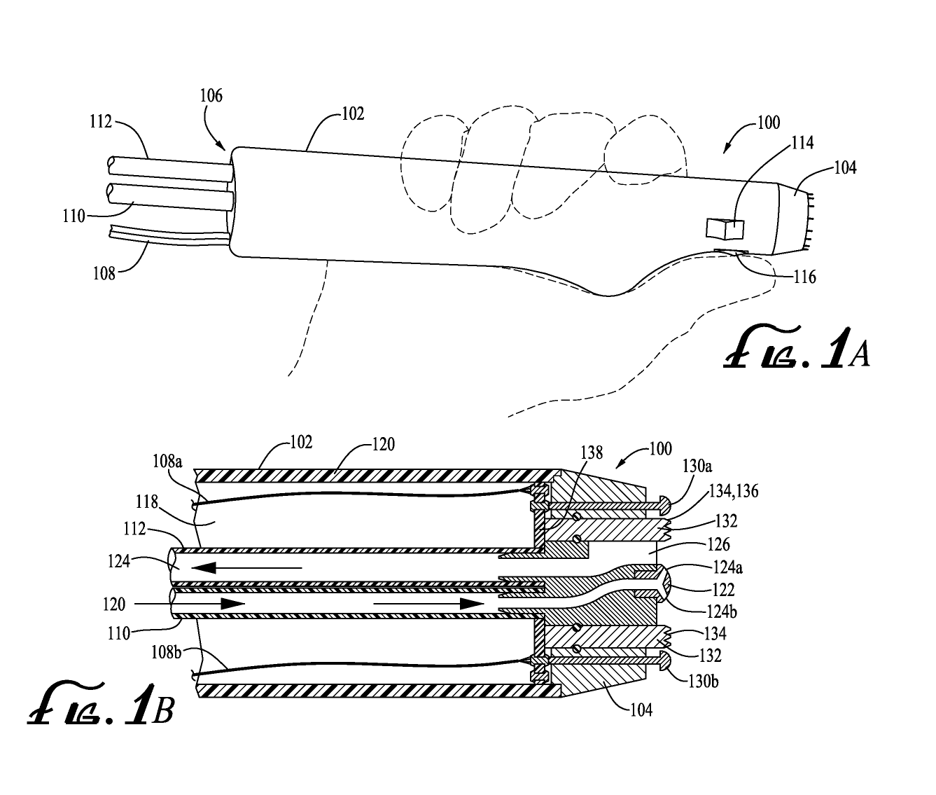

In one embodiment, the present invention is a device for enhancing fluid delivery to the skin. Referring now to FIG. 1A, a skin abrading device 100 having fluid and current delivery is shown. The device 100 comprises a handle 102, a tip 104, and a distal end 106. Positioned at the distal end are one or more conduits such as an electrical conduit 108, a fluid delivery conduit 110, and a vacuum conduit 112. The skin abrading device 100 may further include one or more switches for controlling the device 100 such as a switch 114 and/or 116 for controlling electrical current delivered via the electrical conduit 108, and/or control vacuum and/or fluid delivery from the fluid delivery and vacuum conduits 110 and 112. However, in other embodiments, these switches are positioned remotely on an adjunct device. The optional vacuum function of the device evacuates fluid and skin debris from the surface of the skin and delivers the evacuated fluid and skin debris to an optional waste container (not shown) which may be positioned on the handle or in an adjunct device.

As shown in FIG. 1A, the handle 102 may be cylindrical with molded hand grip, or it may have other configurations such as cylindrical (without a molded hand grip), or other variations, including elliptical, square, rectangular, and variations thereof. The handle 102 may be formed of various materials as known to those in the art including any suitable plastic, metals, such as aluminum, stainless steel, and other alloys, and combinations of metal and plastic. Preferably, the handle 102 is made from a high density plastic material.

Referring now to FIG. 1B, a partial side cut-away view of the device 100 shown in FIG. 1 is shown. As shown in FIG. 1B, the handle 102 of the device 100 comprises an interior 118 and an outer casing 120. The fluid delivery conduit 110 is positioned in the interior 118 of the handle 102 and delivers fluid 120 from a reservoir (not shown) in an adjunct device through the fluid delivery conduit 110 and out the tip 104 of the device 100. The fluid 120 exits the tip 104 through a fluid delivery tip 122 having one or more apertures 124. Also positioned within the interior 118 of the handle 102 is the vacuum conduit 112 which pulls a vacuum from a vacuum pump (not shown) stationed in an adjunct device through the vacuum conduit 112. The vacuum conduit 12 has a vacuum entry port 126 positioned within the tip 104 for evacuating fluids and other debris from the surface of the skin. The interior 118 of the device 100 has one or more electrical conduits 108a, 108b, which deliver current either to an electronics board 128, which then delivers current to one or more electrodes 130, shown as 130a and 130b. Positioned within the tip 104 is an abrading structure 132 having an abrading end portion 134, which comprises an abrasive media 136. Within either the interior 118 of the device, electronic control circuitry 138 may be positioned for controlling current to the electrodes 130.

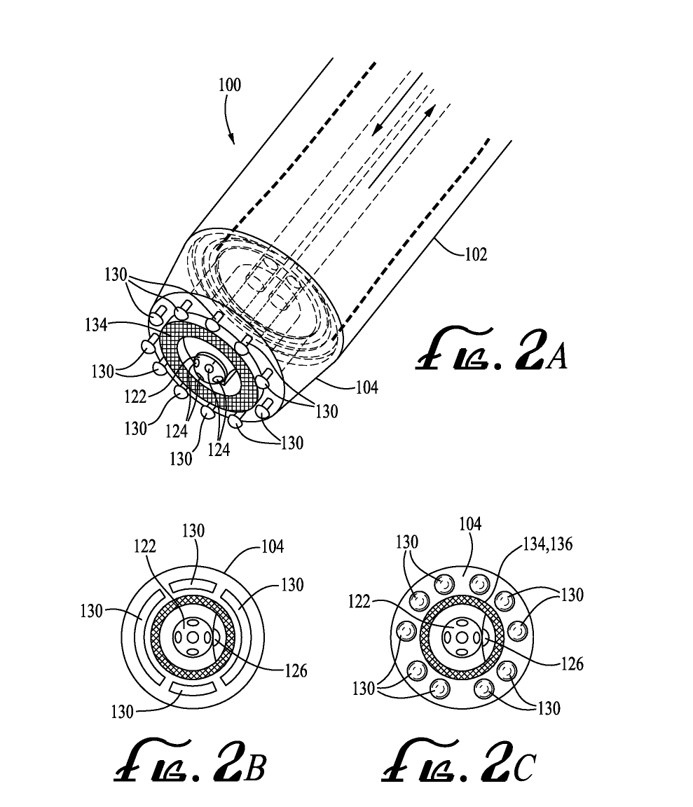

Referring now to FIGS. 2A, 2B, and 2C, preferred embodiments of the tip 104 of the device 100 are shown. As shown in FIG. 2A, the tip 104 may be somewhat tapered at the end, or in other embodiments, the tip 104 may be substantially cylindrically shaped or other, such as oval shaped, squared, or rectangularly shaped. As also shown in FIG. 2A, preferably, the fluid delivery tip 122 is domed shaped, having a plurality of apertures 124, such that a spray effect is achieved with the fluid delivery tip 122. (However, in other embodiments, the fluid delivery tip 122 may be flat, and/or have a single aperture 124. Preferably, the fluid delivery tip 122 is positioned with respect to the tip 104, electrodes 130, and abrading structure 132 such that the fluid delivery tip extends slightly beyond or substantially flush with the abrading structure 132.

The vacuum entry port is positioned with respect to the tip 104, such that the vacuum entry port 126 minimizes skin trauma and ruptured capillaries, veins and arteries from the vacuum 124, yet creates a suitable vacuum to evacuate fluid and debris from the skin's surface. According to a preferred embodiment, the vacuum entry port 126 is positioned on the tip 104 such that when the tip 104 of the device 100 is applied to the surface of the skin, a space is created between the tip 104 and the vacuum entry port 126 to create a vacuum, known in the art as a closed loop system.

In a preferred embodiment, the fluid delivery tip 122 is substantially flush to the skin with respect to the abrading end portion 134 of the abrading structure 132 and the electrodes 130 such that when the device 100 is applied to the skin, the skin stays relatively flat during treatment. According to this embodiment, when the abrasive media 136, vacuum 124, fluid 120, and electric current 140 are applied to the skin with the configuration described with respect to this embodiment, having the various structures of the tip 104 substantially flush to the skin minimizes the possibility of skin trauma associated with the pulling up of skin in a space of vacuum 124.

In an alternate embodiment, the vacuum entry port 126 can be positioned in other portions of the tip 104 to provide an optimal vacuum of concurrent liquid delivery and/or removal of skin debris. However, the vacuum entry port 126 is preferably positioned to keep a higher level of fluid within the tip of the handle during treatment so as to have a higher absorption and penetration rate of ingredients contained in the fluid, into the skin, while still evacuating skin debris and preventing the fluid 120 from flowing away from the desired treatment area and/or falling off the skin.

The abrading structure 132 is positioned with respect to the tip 104, such that the abrading end portion 134 of the abrading structure 132 is substantially flush to the surface of the skin. In other embodiments, the abrading structure 132 may be lowered or raised with respect to the end of the tip 104 to provide skin contact, as desired by the user.

In a preferred embodiment, the abrading structure 132 has a range of abrasiveness on the abrasive media 136 from a substantially smooth surface (no abrasion) to very abrasive depending on the treatment type. As shown in FIGS. 1B, and 2A-2B, the abrading structure 132 is positioned on the outer edge of both a fluid supply, i.e., the fluid delivery tip 122 and vacuum port 126 and on the inside of the electrodes 130. However, according to the present invention, other arrangements of the abrading structure 132, electrodes 130, and fluid delivery tip 122 and vacuum port 126 are possible, as will be understood by those of skill in the art.

The abrading structure 132 may be reusable or disposable, in part or entirely. For example, according to one embodiment, the abrading end portion 134 and the abrasive media 136 are integral to the abrading structure 132. According to this embodiment, the abrading structure may be reusable or disposable in part or entirely. When the abrading structure 132 is reusable, it is preferably designed to be sanitized and cleaned between uses and reused. In an alternate embodiment, the abrasive media 136 is positioned on the abrading end portion 134 in a removable fashion, such as a removable strip. According to this embodiment, the abrading structure 132 is generally reusable and the abrasive media 136 on the abrading end portion 134 is preferably disposable.

The abrasive media 136 comprises a material suitable to abrade the surface of the skin such as sand paper, rough textiles (such as denial grade fabrics that are used in cosmetic microdermabrasion, typically made from 100% medical grade nylon and have a plurality of coatings and finishes), wire brushes, carbon fibers, and microneedles. The material can be conductive or nonconductive. According to one embodiment, the abrasive media 136 comprises a non-conductive sand paper. In one embodiment, the sand paper is white aluminum oxide, a non-conductive material, readily available at low cost in medical grade. This material is able to withstand elevated temperatures, such as those typically present in any vitrification process that may be necessary for high volume binding/fabrication to produce the abrasive tip. According to other embodiments, a material softer than aluminum oxide is preferred so that the material is less irritating to the skin than aluminum oxide, According to this embodiment, the abrading media 136 comprises polymeric beads. Generally, polymeric beads provide a softer, less irritating material than aluminum oxide. However, other materials according to the invention may be used as the abrading media 136, where the material is selected based on the particular individual to be treated and the purpose of the treatment. Accordingly, for different individuals, different materials may be substituted for the above-listed materials. In other embodiments, the abrasive media 136 comprises a conductive material. Suitable conductive materials include, but are not limited to, metals, carbon, conductive polymers and conductive elastomers

The abrading end portion 134 may have a variety of suitable thicknesses and diameters. According to one embodiment, abrasive particles are coated onto the abrading end portion of the abrading structure 132. In some embodiments, the abrading structure 132 and abrading end portion 134 comprise a unitary plastic structure, such as acrylonitrile butadiene styrene (ABS). According to this embodiment, the abrasive media is an abrasive coating adhered to the abrading end portion 132, or the abrasive media 136 is of a unitary construction with the abrading structure 132 and abrading end portion 134. According to one embodiment, the abrasive media comprises abrasive particles which are adhered to the abrading end portion 134, where the thickness of the abrasive media 136 is defined by the grit size of the abrasive particles. According to this embodiment, the abrasive particles are generally of a size ranging from about 300 to 50 grit (about 50 to 300 microns), and typically about 100 to 120 grit and may comprise carborundum (aluminum oxide), sodium bicarbonate, polymeric particles, and the like. Coarser particles (at the lower ends of the grit ranges (about 35 to 50, and typically less than 100) may also be provided for use in initial treatments, or treatments on coarser areas of the skin (such as arms), while finer particles (at the higher ends of the grit ranges about 300 and above) may be employed for subsequent treatments. Alternately, the abrading end portion 134 may be formed by knurling, machining, laser treatment or otherwise mechanically or chemically treating the end of the abrading end portion 134 to provide an integral abrasive media 136 which has a unitary construction with the abrading end portion and abrading end structure 132. In a preferred embodiment, the abrasive media 136 is abrasive particles having a grit size of about 120 or lower (approximately 0.0044 inches in diameter).

Typically the abrading end portion 134 will have a thickness ranging from 0.5 microns to 150 microns, preferably ranging from 15 microns to 120 microns. The diameter of the abrading end portion 134 is variable depending on the type of application, For example, in applications having a small area to be permeabilized, the abrading end portion 134 can have a diameter of up to several micrometers, such as from 1 to 25 microns. For applications having a larger area to be permeabilized, the abrading end portion 134 can have a diameter of up to several inches, such as from 0.1 to 5 inches (2.5 mm to 127 mm).

According to the present invention, a current 140 (not shown) is delivered from the device 100 to the surface of the skin through one or more electrodes 130. The electrodes 130 can be a single electrode, or a plurality of nodes or combination thereof, and may further have a variety of configurations and dimensions, such as nodes, bars, etc., as will be understood by those of skill in the art.

Electrical currents, known for application to the skin, which may be used according to the present invention include:

a. Electroporation. Electroporation refers to the application of electric pulses to increase the permeability of cell membranes. According to the present invention, electric pulses are applied to skin cells to increase membrane permeability.

b. Microcurrent. Microcurrent refers to the application of a small current used in a noninvasive electrotherapy technique where electrodes are applied at acupuncture points. In general, 10-500 microamps (Ua) are applied to the surface of the skin and for optimal effectiveness, the current applied to the skin should not cause an actual “visual” contraction of the facial muscles. In some applications, electroporation refers to the process of applying a microcurrent to the surface of the skin.

c. Iontophoresis. Iontophoresis refers to a therapeutic type of transcutaneous drug delivery in which electric current is applied to the skin to enhance absorption of large polar or hydrophilic molecules and peptides—e.g., insulin, and control therapeutic delivery. According to the present invention, a galvanic current is applied an ionizable agent in contact with a surface of the skin, by means of an appropriate electrode, to hasten the movement into the tissue of the ion of opposite charge to that of the electrode. Accordingly, skin enhancing agents which are polar or hydrophilic may be delivered into the skin.

d. Sonophoresis. Sonophoresis refers to a process that exponentially increases the absorption of semisolid topical compounds (transdermal delivery) into the epidermis, dermis and skin appendages. Sonophoresis occurs where ultrasound waves stimulate micro-vibrations within the skin epidermis and increase the overall kinetic energy of molecules making up topical agents. Skin enhancing agents may be mixed with a coupling agent (gel, cream, ointment) to transfer ultrasonic energy from the ultrasound transducer (i.e., electrode) to the skin and enhancing drug transport through the skin.

e. Galvanic. Galvanic or Galvanic current refers to the current which is the electrical current used in the process of Iontophoresis.

f. Ultrasound. Ultrasound or ultrasonic current refers to the current used in Sonophoresis. Ultrasound is cyclic sound pressure with a frequency greater than the upper limit of human hearing. Although this limit varies from person to person, it is approximately 20 kilohertz (20,000 hertz) in healthy, young adults and thus, 20 kHz serves as a useful lower limit in describing the ultrasonic current applied via the electrodes in the present invention.

g. Ultrasonic Cavitation. Ultrasonic Cavitation refers to an advanced ultrasonic machine having 3 MHz and 1 MHz ultrasound frequencies for the body and a 1.4 MHz ultrasonic frequency for the face, and an ultrasonic cavitation wavelength at 47 KHz. In Ultrasonic Cavitation, the ultrasonic waves are able to act on the skin surface (3 MHZ ultrasound), providing skin tightening as well in the deep layers, (cavitation) providing real results, after the treatment, in terms of cellulite, and localized adiposity. It has been shown to be able to eliminate centimeters of belly, buttocks, hips and thighs without any side effects. Ultrasonic waves in a specific range from 20 to 70 KHz are able to cause the “cavitation” effect: focused high energy waves which creates micro bubbles of vapour inside the adiposities and in the interstitial liquids of cellulite.

h. Acoustic Cavitation. Acoustic Cavitation refers to a non-flowing system where the ambient pressure can be varied by sending sound waves through a liquid. The ultrasonic sound waves are made up of alternate compressions and rarefactions. During the rarefaction cycle (low pressure) a lot of microscopic bubbles will grow and during the compression cycle (high pressure) each bubbles undergoes a collapse or implosion.

i. Mesotherapy. Mesotherapy refers to a procedure in which multiple tiny injections of pharmaceuticals, vitamins, etc., are delivered into the mesodermal layer of tissue under the skin, to promote the loss of fat or cellulite.

J. Radio Frequency. Refers to a procedure using a beam of radio frequency energy to target deeper layers of the skin by heating them up. This creates stimulation of the skin and in particular, the collagen, a substance which gives elasticity to the skin. The radio frequencies cause water molecules in the deeper layers of skin to vibrate. This in turn creates friction which causes the heating effect. When heat is applied to collagen fibers, they shrink and tighten up, and over time following the treatment, new collagen also forms.

k. Hot and cold therapies. Refers to using an electrical current and other modalities to create different adjustable temperatures ranging from hot (up to 140 degrees Fahrenheit) to cold (down to 5 degrees Fahrenheit) to treat the surface layer skin by softening and/or tightening collagen fibers.

In a preferred embodiment of the present invention, a microcurrent is applied to the skin, i.e., electroporation. According to this embodiment, the current of the device 100 is set for a wave form with power between 10-500 microamps (Ua). The current 140 (not shown) is delivered through the device 100 and through one or more electrodes 130 to the surface of the skin. Treatment can be substantially stationary in certain areas, or vary in the degree of motion, up to sweeping lines.

According to another embodiment, a combination of two or more frequencies of current are applied from the device 100 to a patient. Accordingly, in some embodiments the device is capable of delivering a plurality of different frequencies (i.e., types) of current, either individual applied or concurrent. For example, an ultrasonic current may be applied from the device 100 to a patient, followed by delivery of a microcurrent from the device 100 to the same patient. The treatment may be in one treatment area, or over a plurality of treatment areas, such the delivery of microcurrent to the face, followed by delivery of ultrasonic current to the arms. The plurality of frequencies may be used on one patient for application of different electric currents. For example, ultrasound and microcurrent have different ways of penetrating fluids and treating the skin. The concurrent combination of these and other electric modalities shown in device 100 is to provide a more effective treatment.

Referring again to FIG. 1B, fluid 120 is delivered from a fluid reservoir (not shown), which may be either part of the handle or in a separate reservoir, such as a plastic or glass tube serum, through the fluid delivery conduit 108 and out the fluid delivery tip 122 in the tip 104 of the device 100. Fluid delivery may be used in the device for cleaning of the skin, as a vehicle for delivery of a therapeutic agent, or it may be the therapeutic agent itself, and/or the fluid may be an ionic agent to facilitate delivery of current 140 through the electrodes 130. The fluid may include one or a plurality of suitable skin enhancing agents, and/or conductive ingredients, or other suitable agents for skin cleaning and skin enhancement or facilitation of current delivery, such as water, salts, ionic or non-ionic surfactants, preservatives, alcohol, glycerol, gel, and other similar agents. Various mixtures of these agents may be formulated into fluids with various conductivity levels, depending on the desired application. Preferably, at least one of the fluids used in a method according to the present invention is a “highly conductive fluid” or a “fluid with a high conductivity” meaning a fluid with a conductivity from about 1,000 to about 100,000 (μSiemens/cm) to facilitate current delivery. Other fluids, such as a “fluid with a low conductivity”, meaning a fluid with a conductivity from about 0.1 to about 999 (μSiemens/cm), are used according to the invention in other applications, such as cleaning, and/or delivery of a skin enhancing or therapeutic agent. A highly conductive fluid is used according to the present invention to provide a conductive path through the skin. In a preferred embodiment, at least one fluid with a conductivity of at least 500 to about 50,000 uSiemens/cm is used,

Therapeutic or skin enhancing fluids useful in the device 100 according to the present invention may be of a variety of therapeutic agents. For example, the fluid may be a skin treatment liquid, a lotion liquid, and/or a vitamin liquid, or a combination thereof. The fluid may also be a pharmacologically-active agent, where the fluid carries a chemical agent of a suitable concentration, Examples of such agents include TCA (trichloroacetic acid), a glycolic acid including an alphahydroxy acid (AHA), a lactic acid, a citric acid, and phenol, alone or in combination with other agents or fluids. Examples of other therapeutic or skin enhancing agents include type A botulinum toxine, phosphatidylcoline, aminophylline, hyaluronic acid, L-carnitine vitamins, amino acids, collagen, lidocaine, heparin, elastine, compounds for Mesotherapy procedures, glutathione, hormone replacement agents, hyaluronidase, MTE-4 (Copper-Manganese-Zinc Sulphate-Chromium), ionic skin tissue growth gels, enzymes, peptides and steroids.

Other ingredients can include plant and fruit derived ingredients, such as enzymes and stem cells derived from fruits and/or plants, etc. Since microdermabrasion is a controlled injury of the skin by abrading the surface layer to cause a wound healing response, other known healing and anti-inflammatory ingredients such as cortisone, aloe extract, etc. may be used to increase healing response time and also act as an anti-fungal, anti-viral, anti-bacterial and acaricidal activity against skin infections such as acne, etc. may be used individually or in any combination with other sterile fluids, drugs, and other skin enhancing and/or therapeutic agents.

Other agents and preferred viscosity parameters may be found in “Advanced drug delivery reviews”, 56 (2004) 659-674.

Referring again to FIG. 1B, a vacuum 124 may be applied to the surface of the skin from a vacuum pump (not shown) through the vacuum conduit 112 and vacuum entry port 126 on the tip 104 of the device. Preferably, the vacuum pump which supplies the vacuum 124 to the device 100 has a rating of 2.9A, with a max flow rate of 2 cu.ft/min, a power rating of 120 W, with a 60 Hz frequency, and preferably RoHS compliant, although other embodiments are possible. In general, the vacuum 124, used during a treatment and applied to the surface (or just above) the skin of a patient, is a continuous flow and preferably can be adjusted with a flow control valve to increase or decrease vacuum pressure.

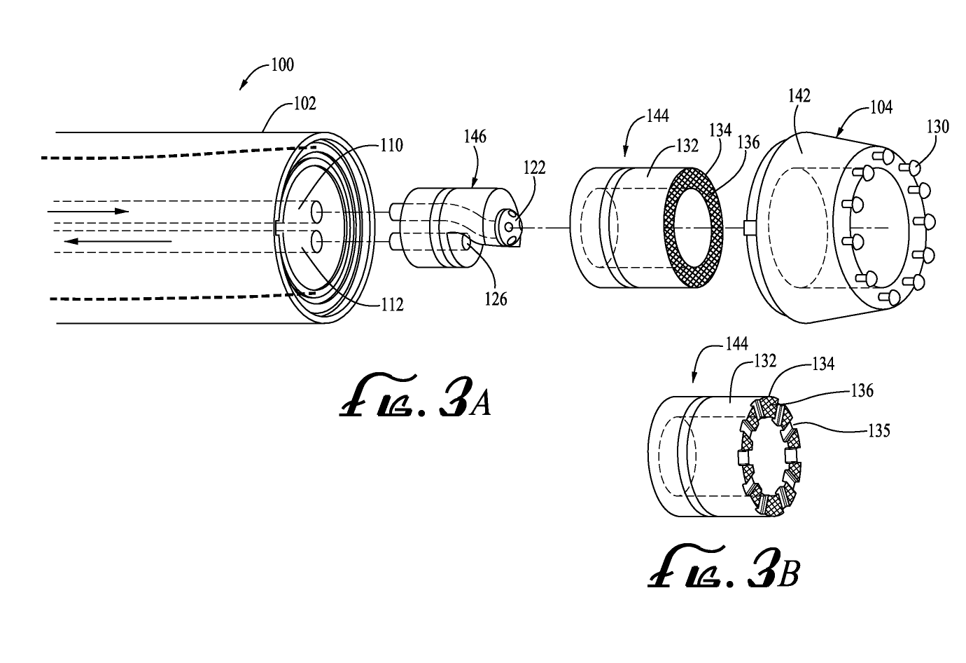

Referring now to FIG. 3A, a skin abrading device 100, having a plurality of removable, exchangeable, and attachable tips, according to a preferred embodiment of the invention is shown. As shown in FIG. 3A, the tip 104 of the device 100 comprises multiple nesting (e.g., interconnected) structures which are removable/attachable from the handle 102. The outer structure 144 of the tip 104 comprises the electrodes 130 at the proximal end of the tip 104 and wiring (not shown) for delivering current 140 (not shown) to the electrodes 130. Positioned within the outer structure 144, is the intermediate structure 144, which is also the abrading structure 132. The inner structure 146 comprises the fluid delivery tip 122 and vacuum entry port 126. When the structures 142, 144, and 146 (i.e., tips) are assembled, the tip 104 of the device 100 will have the configuration shown in FIGS. 1A, 1B, and 2A-2C. The outer structure 142, intermediate structure 144 and inner structure 146 are connected to the handle 102 with a suitable connection, such as compression fitting, threaded fittings, etc. In a preferred embodiment, one or more of the outer structure 142, intermediate structure 144, and inner structure comprise stainless steel. In one preferred embodiment, the intermediate structure 144 comprises a reusable stainless steel abrading structure 132 having an abrading end portion 134 which has a diamond coated abrasive as the abrasive media 136. In another preferred embodiment, the intermediate structure 144 comprises a disposable (preferably translucent) plastic abrading structure 132 having a disposable abrasive media 136 positioned on the abrading end portion 134. In another preferred embodiment, the inner structure, comprising the fluid delivery tip 122 and the vacuum entry port 126, are one or more of transparent, detachable, and/or disposable. Although the outer structure 142, intermediate structure 144, and inner structure 146 have been described herein as removable, exchangeable, and attachable, it will be understood by those of skill in the art that one or more of the outer structure 142, intermediate structure 144, and inner structure 146 may be affixed to the handle 102 in a permanent, or not-easily removable fashion. However, in other embodiments, one or all of the structures 142-144 may be one piece in any arrangement or separate individual connections. For example, the device may comprise a handle 102 with an electric current node (i.e., electrode 130) in the middle surrounded by a fluid delivery piece 122 and an abrasive structure 132 making the outer edge of the handle. This is just an opposite arrangement from the arrangement shown in FIG. 3A, and as it will be understood by those of skill in the art, interrelationship of the various tips shown in the Figures is by way of example and other configurations are within the scope of the invention.

Referring now to FIG. 3B, another embodiment of the abrading structure 132 is shown. According to this embodiment, the abrading end portion 134 of the abrading structure 132 comprises one or more grooves 135. The grooves may be differently shaped, such as rounded grooves, or slotted squares. The grooves 135 are provided to abrade the skin more effectively by stretching it, and to better guide skin debris into the vacuum. Preferably, to keep the vacuum 124 sealed, the grooves 135 are substantially even with the edge such that when the abrading structure 132 is applied to the skin, air does not escape. The grooves 135 may have a variety of thickness or radius, shape or design, for different skin types and applications, as will be understood by those of skill in the art. According to this embodiment, extraction can be realized by pressing the abrading end portion 134 and grooves 135 to the skin, such that the grooves 135 act as a comedo extractor on a pore. For example, when the abrading end portion 134 having grooves 135 is pressed to the skin, oil and sebum will be released from the pores.

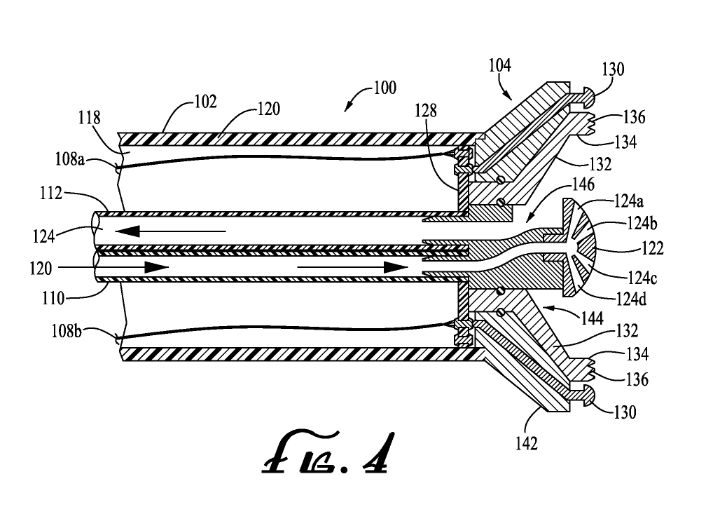

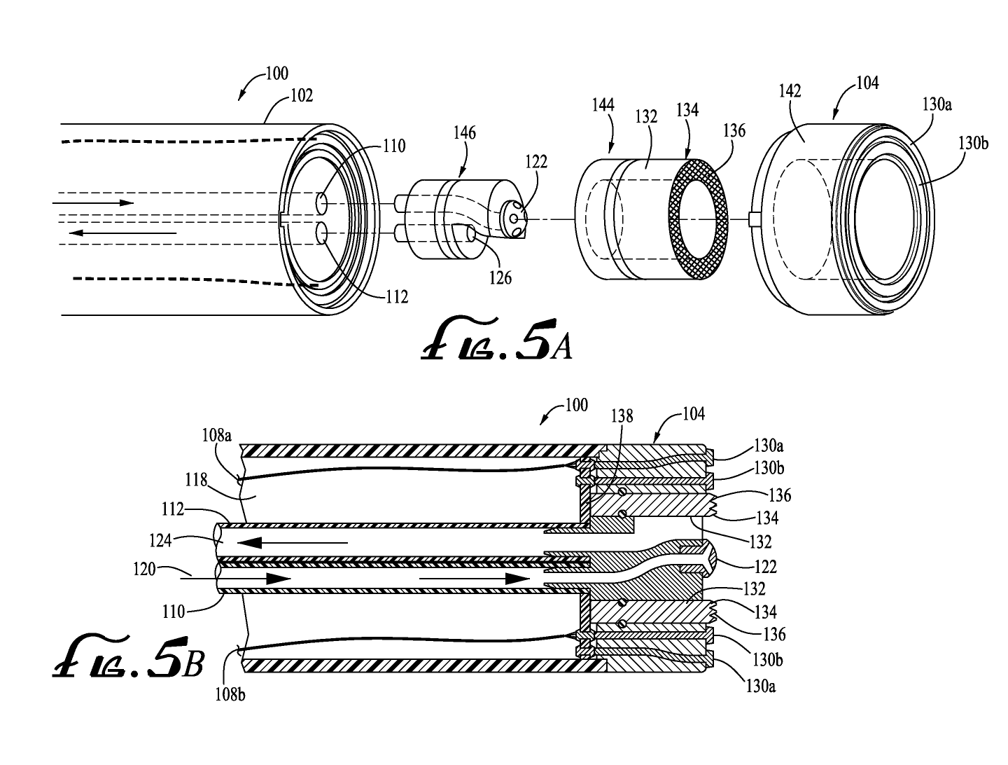

Referring now to FIG. 4, a partial side cut-away view of the device 100 having a wide-angle tip 104 is shown. As shown in FIG. 4, the same numbers refer to the same features shown in FIG. 1B, with the differences noted below. According to this embodiment, the tip 104 of the device is a wide angle tip, where the fluid 120 exits the tip 104 through a fluid delivery tip 122 having a plurality of apertures 124a-124d. According to this embodiment, the wide angle tip allows for an increased area for fluid delivery and more apertures for fluid delivery. The interior 118 of the device 100 has one or more electrical conduits 108a, 108b, which deliver current either to an electronics board 128, which then delivers current to one or more electrodes 130, or directly to the electrodes. As the tip 104 is a wide-angle tip, the electrodes are positioned further from the center of the tip 104 and in some embodiments, this allows for additional or wider electrodes 130 than the tapered tip 104 shown in FIG. 1A and FIG. 1B. Positioned within the wide angle tip 104 is an abrading structure 132 having an abrading end portion 134, which comprises an abrasive media 136. Similarly to the electrodes 108, the abrading end portion 124 and abrading media 132 are positioned further from the center of the device than the tapered tip 104 shown in FIG. 1A and FIG. 1B. This embodiment may be used on a treatment area with a larger surface area that can accommodate the larger tip surface area. The various tips comprising the outer structure 142, intermediate structure 144, and inner structure 146, shown in FIG. 4, may be removable, exchangeable, and attachable, and may be exchanged with other interchangeable tips 142-144, of other dimensions, as described herein. Referring now to FIG. 5A, a skin abrading device 100, having a plurality of removable, exchangeable, and attachable tips, according to another preferred embodiment of the invention is shown. Unless otherwise noted below, the same reference numbers refer to the same elements as described with reference to FIG. 3. As shown in FIG. 5A, the tip 104 of the device 100 comprises multiple nesting (e.g., interconnected) structures which are removable/attachable from the handle 102. As shown in FIG. 5A, the electrodes 130a and 130b, are concentric circles positioned within the outer structure 144 of the tip 104. Positioned within the outer structure 144, is the intermediate structure 144, which is also the abrading structure 132. The inner structure 146 comprises the fluid delivery tip 122 and vacuum entry port 126. Referring now to FIG. 5B, a partial side cut-away view of the device 100 shown in FIG. 5A, having electrodes 108a, 108b, which are concentric circles is shown. When the structures 142, 144, and 146 (i.e., tips) are assembled, the tip 104 of the device 100 will have the configuration shown in FIG. 5B. The outer structure 142, intermediate structure 144 and inner structure 146 are connected to the handle 102 with a suitable connection, such as compression fitting, threaded fittings, etc. As shown in FIG. 5A and 5B, the tip 104 is substantially linear with respect to the handle. However, in other embodiments, the tip 104 may be tapered as shown in FIG. 1A or wide-angled, as shown in FIG. 4. The structures 142-146 may comprise any suitable metal such as stainless steel, or may be any suitable plastic that is transparent, detachable, and/or disposable, and may be removable, etc. as shown in FIG. 5A, or substantially fixed, as described herein with respect to other embodiments, as win be understood by those of skill in the art.

Although the electrodes 130, shown in FIG. 5A and other Figures, are shown as positioned on the outer structure 144, the electrodes 130 may be positioned on the inner structure 146 and the fluid delivery portion 122 and/or the abrasive portion 132 may be positioned in the outer and intermediate structures 142 and 144, in a variety of combinations, either removable/attachable or permanently part of the handle, as will be understood by those of skill in the art.)

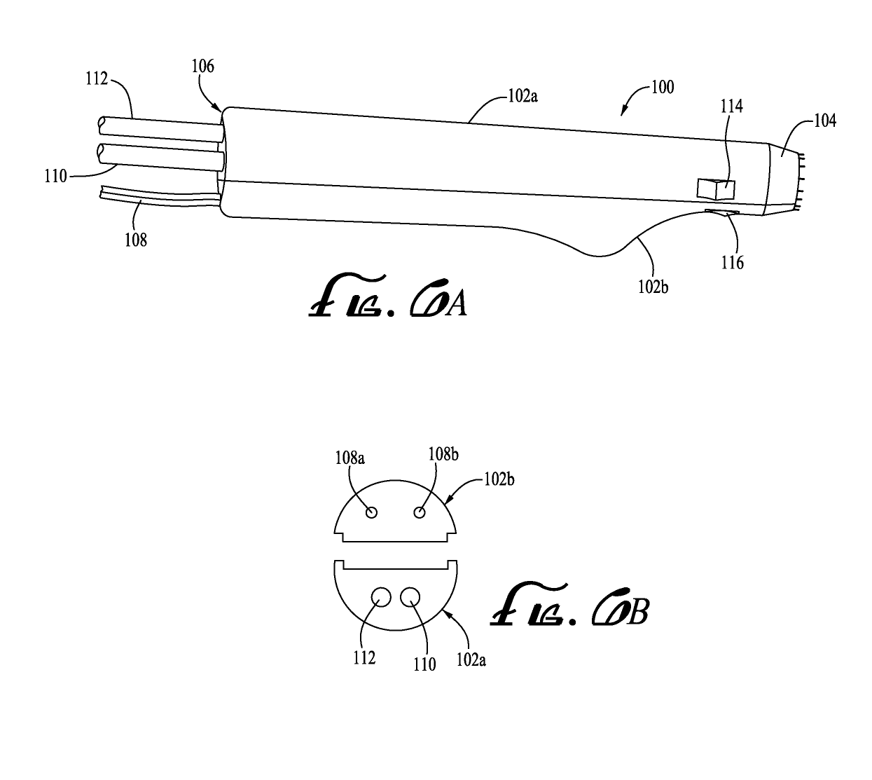

FIG. 6A shows an alternate embodiment of the skin abrading device 100 according to another embodiment of the present invention. As shown in FIG. 6A, the device 100 has a divided handle 102a and 102b. FIG. 6B is a cross sectional view showing the divided handle 102a and 102b of FIG. 6A. As shown in FIG. 6B, the top portion of the handle 102a comprises the fluid delivery conduit 110 and the vacuum conduit 112 and the bottom portion of the handle 102b comprises the electrical conduits 108a. The tip 104 of the device 100 shown in FIG. 6B, may have one or all of the configurations disclosed herein, including removable/interchangeable outer, intermediate and inner structures 142, 144 and 146 for the tip 104 portion of the device 100, as shown in FIGS. 3-5.

As shown in FIGS. 1-6, each of the embodiments described comprises tip 104 having electrodes 130, an abrading structure 132, and fluid delivery 122. However in other embodiments, the device may have only two of these features, such as the combination of electrodes 130 and fluid delivery 122, without the electrode 130 feature, as will be understood by those of skill in the art.

According to another embodiment, a method for treating a skin surface of a patient is provided. According to the method, a device according to the invention is employed to abrade the skin surface of a patient; deliver fluid to the surface of the skin; and apply current to the surface of the skin. These steps may be performed in the sequence described herein, or the sequence may be altered, depending on the type of procedures to be performed on the patient, as will be understood by those of skill in the art.

In a preferred embodiment, first the abrading end portion 134 of the abrading structure 132 of the device 100 is applied to the skin surface of a patient. Vacuum may optionally be applied to the skin surface to remove any residual debris, such as abrasive media and excess skin, either after or during the abrading portion of the treatment. Then, the skin surface is contacted with the abrading end portion 134 and abrasive media 136 of the device and the abrading end portion 134 of the device 100 is moved over the surface of the skin. Treatment can be substantially stationary in certain areas, or vary in the degree of motion, up to sweeping lines. Next, a fluid is provided to the skin surface through the fluid delivery tip 122 of the device 100. Then, a current 140 is applied to the surface of the skin by transferring current from the electrodes 130 to the skin surface. The current 140 may be applied either to wet or dry skin.

Although the method is described above as being performed in a sequential manner, this is provided by way of example, and is only one of the possible protocols for the method of the invention. Accordingly, according to the method of the invention the various treatments, including skin abrasion, fluid delivery, and/or current delivery may be performed concurrently, or one at a time, in any order, depending on the patient needs and treatment given to any particular patient.

Although the present invention has been discussed in considerable detail with reference to certain preferred embodiments, other embodiments are possible. Therefore, the scope of the appended claims should not be limited to the description of preferred embodiments contained herein.

What is claimed is:

1. A device for treating a skin surface of a patient comprising:
a handle,
a tip at the proximal end of the handle, the tip comprising one or more electrodes;
an abrading end portion having an abrasive media; and
one or more apertures for fluid delivery.

2. The device according to claim 1 wherein the tip further comprises a vacuum entry port having one or more apertures.

3. The device according to claim 1 wherein the tip comprises one or more structures, and wherein at least one of the structures is removable.

4. The device according to claim 1 wherein the tip comprises:
an outer structure having one or more electrodes;
an intermediate structure having an abrading end portion, the abrading end portion comprising an abrasive media; and
an inner structure comprising one or more apertures for fluid delivery.

5. The device according to claim 4 wherein at least one of the structures is removable.

6. The device according to claim 4 wherein each of the outer structure, intermediate structure, and inner structure are removable.

7. The device according to claim 4 wherein each of the outer structure, intermediate structure, and inner structure are removable and at least one of the structures is disposable.

8. The device according to claim 4 wherein the abrading end portion comprises a disposable abrasive tip.

9. A method for treating a skin surface of a patient comprising:
(a) selecting an abrasion device for treating a skin surface of a patient, wherein the abrasion device comprises one or more electrodes, an abrading end portion having an abrasive media, and one or more apertures for fluid delivery;
(b) placing the abrading end portion of the device on the skin surface of the patient;
(c) applying the abrasive media on the abrading end portion of the device to the skin surface of the patient;
(d) delivering fluid to the skin surface of the patient; and
(e) applying an electrical current to the skin surface of the patient.

10. The method of claim 9 further comprising selecting a treatment area of a patient; and performing each of steps (b)-(c) on the skin surface of the treatment area of the patient.

11. The method of claim 9 further comprising applying a vacuum to the skin surface of the patient.

12. The method according to claim 9 wherein the fluid delivered to the skin is a therapeutic or skin enhancing agent.

13. The method according to claim 9 wherein the fluid delivered to the surface of the skin comprises an ionic agent to facilitate delivery of current to the skin surface.

14. The method according to claim 9 wherein the current applied to the skin surface is one or more of microcurrent, galvanic current, ultrasonic current.

15. The method according to claim 9 wherein the current applied to the skin surface comprises an electrical frequency or wave form having a therapeutic effect on the skin.

16. The method according to claim 9 wherein the abrasive media on the abrading end portion of the device has a grit of between about 100 to 120.

17. The method according to claim 9 wherein the abrasive media on the abrading end portion of the device is substantially smooth, having a grit of about 300 or greater.

18. The method according to claim 9 wherein the abrasive media on the abrading end portion of the device has a grit of about 50.

19. A kit for treating a skin surface of a patient comprising:
a skin abrading device comprising a tip, wherein the tip comprises:
at least one current delivery tip having one or more electrodes,
a plurality of abrading tips, wherein each abrading tip has an end portion with an abrasive media, and wherein the plurality of abrading tips are removable from the device and interchangeable; and
a fluid delivery tip having one or more apertures for fluid delivery.

20. The kit according to claim 19 wherein the tip further comprises a vacuum entry port.

21. The kit according to claim 19 wherein each of the plurality of abrading tips has a grit size, and the grit size varies for each abrading tip.

22. The kit according to claim 19 wherein the grit size for each abrading tip varies from between 50 microns to 300 microns.

23. The kit according to claim 19 wherein the one or both of the current delivery tip and the fluid delivery tip are removable.

24. The kit according to claim 19 further comprising a second current delivery tip, wherein the current delivery tip and the second current delivery tip are removable and interchangeable.

25. The kit according to claim 19 further comprising a second fluid delivery tip, wherein the fluid delivery tip and the second fluid delivery tip are removable and interchangeable.
